SEQUENCE LISTING (7 sequences)

SEQ ID NO: 1 - DNA, 684 bp, Homo sapiens
  source 1..684: mol_type = genomic DNA
taacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcac

SEQ ID NO: 2 - DNA, 888 bp, Homo sapiens
  source 1..888: mol_type = genomic DNA
atggaacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatctga

SEQ ID NO: 3 - DNA, 19 bp, synthetic construct
  source 1..19: mol_type = other DNA
  ncRNA 1..19: ncRNA_class = other; note = short harpin RNA
gcaagtggtttcgactttc

SEQ ID NO: 4 - DNA, 5408 bp, synthetic construct
  source 1..5408: mol_type = other DNA
  misc_feature 1..5408
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 5 - DNA, 5525 bp, synthetic construct
  source 1..5525: mol_type = other DNA
  misc_feature 1..5525
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcaactagttctagaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagcggcctggaggcttgctgaaggctgtatgctggaaagtcgaaattccacttgcgttttggccactgactgacgcaagtggtttcgactttccaggacacaaggcctgttactagcactcacatggaacaaatgcggccgcaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 6 - DNA, 5525 bp, synthetic construct
  source 1..5525: mol_type = other DNA
  misc_feature 1..5525
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcaactagttctagaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagcggcctggaggcttgctgaaggctgtatgctgaaatcgctgatttgtgtagtcgttttggccactgactgacgactacacatcagcgatttcaggacacaaggcctgttactagcactcacatggaacaaatgcggccgcaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 7 - DNA, 5715 bp, synthetic construct
  source 1..5715: mol_type = other DNA
  misc_feature 1..5715
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcactagtgccacatggaacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatctgagcggcctggaggcttgctgaaggctgtatgctggaaagtcgaaattccacttgcgttttggccactgactgacgcaagtggtttcgactttccaggacacaaggcctgttactagcactcacatggaacaaatgcggccgcaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

(19) United States
(12) Patent Application Publication
(10) Pub. No.: US 2026/0061077 A1
(43) Pub. Date: Mar. 5, 2026

(54) METHODS AND COMPOSITIONS OF TREATING RETINAL DEGENERATIVE DISEASES

(71) Applicant: City University of Hong Kong, Hong Kong (HK)

(72) Inventors: Wenjun XIONG, Hong Kong (HK); Zhifei WU, Hong Kong (HK); Baoshan LIAO, Hong Kong (HK)

(21) Appl. No.: 18/820,216

(22) Filed: Aug. 29, 2024

(51) Int. Cl. A61K 48/00; A61K 38/17; A61P 27/02; C12N 15/113; C12N 15/86

(52) U.S. Cl. CPC A61K 48/0058; A61K 38/1709; A61K 48/0075; A61P 27/02; C12N 15/113; C12N 15/86; C12N 2310/14; C12N 2310/531; C12N 2750/14143

(57) ABSTRACT

A method for treating retinal degenerative diseases in a subject, which involves administering an agonist to selectively elevate cyclin D1 expression in Müller glia (MG) cells, and administering an antagonist to selectively knock down p27^{Kip1} expression in MG cells.

### REFERENCE TO SEQUENCE DISCLOSURE

The sequence listing file under the file name “P3252US00_Sequence Listing.xml” submitted in ST.26 XML file format with a file size of 30.8 KB created on Aug. 27, 2024 and filed on Aug. 29, 2024 is incorporated herein by reference.

### FIELD OF THE INVENTION

The present invention generally relates to ophthalmology fields. More specifically the present invention relates to treating retinal degenerative diseases.

### BACKGROUND OF THE INVENTION

Müller glia (MG) are the last cell type generated by retinal progenitor cells (RPCs) during development, exhibiting a gene expression profile similar to that of late retinal progenitor cells (Jadhav et al., 2009; Roesch et al., 2012). In teleost fish and amphibians, MG respond rapidly to retinal injury by undergoing robust proliferation and regenerating lost retinal neurons from MG-derived progenitor cells (Hamon et al., 2016; Todd and Reh, 2022). In contrast, the proliferative and neurogenic abilities of MG in response to injury in mammals are severely limited, failing to mediate retinal self-repair (Karl et al., 2008).

Recent investigations have achieved notable success in mammalian retinal regeneration by directly converting MG into retinal neurons through the application of single or combined neurogenic transcription factors (Jorstad et al., 2017; Levi et al., 2021; Todd et al., 2022; Yumi et al., 2015). However, this approach may lead to a depletion of the MG population, potentially causing further retinal degeneration, as MG are indispensable for retinal function and homeostasis.

Other studies have shown that the quiescent state of MG can be overridden by manipulating upstream signaling pathways such as Wnt and Hippo (Hamon et al., 2019; Rueda et al., 2019; Yao et al., 2016). Activation of Wnt signaling by forced expression of β-catenin in adult mouse MG promoted spontaneous cell cycle re-entry in uninjured retinas (Yao et al., 2016). Additionally, bypassing the Hippo pathway in mouse MG led to spontaneous re-entry into the cell cycle and reprogramming into a progenitor cell-like state (Hamon et al., 2019; Rueda et al., 2019). These findings suggest that the re-entry of MG into the cell cycle, which is the first step of MG-mediated retinal regeneration in zebrafish, could potentially be unlocked in mammalian retinas.

The cell cycle of MG is primarily regulated by cyclins and cyclin-dependent kinases (CDKs). Cyclins bind to CDKs to form cyclin-CDK complexes, which promote cell cycle progression. During retinal development, the expression of D-type cyclins (cyclin D1, D2, and D3) is tightly regulated (Barton and Levine, 2008; Dyer and Cepko, 2001; Trimarchi et al., 2008). Among these, cyclin D1, encoded by the Ccnd1 gene, is the predominant D-type cyclin in the developing retina and is highly expressed in retinal progenitor cells (RPCs) but absent in differentiated cells (Barton and Levine, 2008; Trimarchi et al., 2008). Mice lacking Ccnd1 exhibit small eyes and hypocellular retinas due to reduced RPC proliferation (Fantl et al., 1995; Sicinski et al., 1995), a deficit that cannot be compensated by Ccnd2 and Ccnd3 (Das et al., 2012, 2009).

Negative regulators of the cell cycle are CDK inhibitors (CDKIs), which include the INK4 family (p16^{INK4a}, p15^{INK4b}, p18^{INK4c}, and p19^{INK4d}) and the CIP/KIP family (p21^{Cip1}, p27^{Kip1}, and p57^{Kip2}) (Reynisdóttir et al., 1995). CDKIs inhibit cell cycle progression by binding to and inactivating the cyclin-CDK complexes (Besson et al., 2008), thereby regulating the proliferation of distinct retinal progenitor cells (Dyer and Cepko, 2000a, 2001; Levine et al., 2000). Specifically, p27^{Kip1} inhibits the cyclin D-CDK complex from entering the S phase. Following acute retinal damage in mice, a very small number of MG re-enter the cell cycle, coinciding with the downregulation of p27^{Kip1} or upregulation of cyclin D1 (Dyer and Cepko, 2000b; Hamon et al., 2019; Rueda et al., 2019; Yao et al., 2016). However, this process is transient, as cyclin D1 expression rapidly returns to basal levels (Dyer and Cepko, 2000; Hamon et al., 2019a; Rueda et al., 2019).

Despite the promising potential of MG for retinal regeneration, several challenges remain in effectively inducing MG proliferation and reprogramming in mammals. One major obstacle is the limited proliferative and neurogenic response of MG to injury in mammalian retinas, which contrasts sharply with the robust regenerative capabilities observed in teleost fish and amphibians. Current methods, such as direct conversion of MG to retinal neurons through neurogenic transcription factors, risk depleting the MG population, leading to further retinal degeneration since MG are essential for retinal function and homeostasis. Additionally, while some studies have demonstrated that manipulating upstream signaling pathways like Wnt and Hippo can induce MG cell cycle re-entry, these approaches have not yet achieved consistent or sustained results in promoting effective MG-mediated retinal repair.

Therefore, the present invention addresses these challenges and provides a more effective strategy for inducing MG proliferation and retinal regeneration without compromising the essential functions of MG.

### SUMMARY OF THE INVENTION

It is an objective of the present invention to provide methods, compositions, or usages to solve the aforementioned technical problems.

In accordance with a first aspect of the present invention, a method of treating retinal degenerative diseases in a subject in need thereof is provided. Particularly, the method includes administering an agonist selectively elevates the expression of cyclin D1 in Müller glia (MG) cells; and administering an antagonist selectively knocks down the expression of p27^{kip1} in MG cells.

In accordance with one embodiment of the present invention, the administration enables delivery to the subject's subretinal space or vitreous.

In accordance with one embodiment of the present invention, the combination of cyclin D1 overexpression and p27^{kip1} knockdown drives MG proliferation and induces partial differentiation towards neuron-like MG cells.

In accordance with one embodiment of the present invention, the retinal degenerative diseases include diabetic retinopathy, age-related macular degeneration (AMD), retinitis pigmentosa, Stargardt's disease, macular hole, and bright light-induced retina damage.

In accordance with a second aspect of the present invention, a composition for treating retinal degenerative diseases in a subject in need thereof is introduced. Specifically, the composition includes an agonist selectively elevates the expression of cyclin D1 in MG cells, an antagonist selectively knocks down the expression of p27^{kip1} in MG cells and a pharmaceutically acceptable addition.

In accordance with one embodiment of the present invention, the agonist and the antagonist are integrated in a gene therapy technique, including a recombinant adeno-associated virus (rAAV), a recombinant adenovirus, a recombinant lentivirus, a recombinant retrovirus, a recombinant vaccinia virus, a recombinant herpes simplex virus, a naked plasmid, a lipid nanoparticle, a peptide-based vector and a polymer-based vector.

In accordance with one embodiment of the present invention, the agonist and the antagonist are integrated in a gene therapy technique, including a rAAV vector for delivering gene therapy to a retinal region.

In accordance with one embodiment of the present invention, the rAAV vector includes:
a promoter DNA sequence obtained from a glial fibrillary acidic protein (GFAP) gene;
a DNA sequence encoding cyclin D1; and
a short hairpin RNA sequence targeting p27^{kip1} gene, wherein the short hairpin RNA sequence is encoded in the 3′ untranslated region (UTR) of the cyclin D1 gene.

In accordance with one embodiment of the present invention, the promoter DNA sequence is at least 85% identical to the DNA sequence of SEQ ID NO: 01.

In accordance with another embodiment of the present invention, the DNA sequence is at least 85% identical to the DNA sequence of SEQ ID NO: 02 and encodes a functional cyclin D1.

In accordance with one embodiment of the present invention, the short hairpin RNA sequence is at least 85% identical to the DNA sequence of SEQ ID NO: 03.

In accordance with one embodiment of the present invention, the rAAV vector stimulates MG cells to overexpress cyclin D1 and knock down the expression of p27Kip1 in the MG cells.

In accordance with another embodiment of the present invention, the pharmaceutically acceptable addition comprises one or more of an excipient, a stability additive, a carrier, a diluent, or a solubilizer.

In accordance with one embodiment of the present invention, the composition is formulated to an administration form that enables delivery to the subject's subretinal space or vitreous.

In accordance with one embodiment of the present invention, the administration form is selected from an immediate-release form or a controlled-release form.

In accordance with one embodiment of the present invention, the composition is delivered through an approach selected from an intravitreal injection, or a subretinal injection.

In accordance with one embodiment of the present invention, the administration form includes an injection form, a hydrogel form, an ultrasonic ocular drug delivery form, a drug-eluting implant form, a nanoparticle-mediated delivery, and an intravitreal microneedle form.

In accordance with one embodiment of the present invention, the rAAV vector is delivered in combination with other neurogenic factors to enhance retinal regeneration.

In accordance with a third aspect of the present invention, a usage of the aforementioned composition for treating a retinal degenerative disease in a subject in need thereof is presented. Specifically, the usage includes administering an amount effective of the aforementioned composition to the subject in need thereof.

In accordance with one embodiment of the present invention, the retinal degenerative disease includes diabetic retinopathy, AMD, retinitis pigmentosa, Stargardt's disease, macular hole, and bright light-induced retina damage.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the invention are described in more details hereinafter with reference to the drawings, in which:

FIGS. 1A-1J depict that simultaneous p27^{Kip1} downregulation and cyclin D1 overexpression drive robust MG proliferation in the uninjured mouse retina, in which FIG. 1A exhibits a schematic illustrating the design of the AAV vectors used in the embodiments, FIG. 1B illustrates the experimental design, FIGS. 1C-1F show the analysis of EdU incorporation including the uninjured mouse eyes injected with AAV-GFAP-mCherry-non target (NT) shRNA (FIG. 1C), AAV-GFAP-mCherry-p27shRNA (FIG. 1D), AAV-GFAP-cyclin D1 (FIG. 1E), and AAV-GFAP-cyclin D1-p27shRNA (FIG. 1F) at P6 and harvested at P18 after 5-day EdU intraperitoneal injection, FIG. 1G depicts the quantification of EdU+Sox9+ cells, FIG. 1H shows the quantification of the percentages of EdU+GFP+ and EdU-GFP+ cells in the area with efficient virus infection in the Glast-CreERT2; Sun1:GFP mouse retina injected with the control or CCA vector, FIG. 1I demonstrates the quantification of the total GFP+ MG in the area with efficient virus infection of the CreERT2; Sun1:GFP retina, and FIG. 1J displays the quantification of EdU+Sox9+ cells in the young (CCA injection at P6, n=13), adult (CCA injection at P28, n=17), and aged retinas (CCA injection at P255-P347, n=7);

FIGS. 2A-2E illustrate that the MG proliferation driven by CCA is self-limiting, in which FIG. 2A depicts the time-course analysis of MG proliferation after CCA injection, FIG. 2B shows the analysis of EdU and BrdU labeled cells, FIG. 2C displays the quantification of the number of EdU+BrdU−, EdU−BrdU+ and EdU+BrdU+ cells, FIG. 2D exhibits the representative retinal sections of Glast-CreERT2; SUN1:GFP mice at 1 week, 3 weeks, and 4 months post CCA injection, and FIG. 2E shows the quantification of MG daughter cell distribution in each retinal layers;

FIGS. 3A-3H depict the scRNA-seq analysis of MG 3 weeks post CCA treatment, in which FIG. 3A is a schematic illustration of scRNA-seq experiment, FIG. 3B shows the UMAP plot of FACS-purified MGs treated with CCA, CCANT, and control virus, with clusters identified by the known marker gene expression, FIG. 3C displays the expression of retinal cell markers in different cell clusters, FIG. 3D shows the separate UMAP plot of the control, CCA, and CCANT groups, FIG. 3E displays the proportion of cell clusters in the control, CCA, and CCANT groups, FIG. 3F is a heatmap of top DEG genes between cell clusters, FIG. 3G presents the violin diagram showing the expression of IFN-7 pathway, MG and Rod genes in different cell clusters, and FIG. 3H displays the feature plots of cell cycle regulator (Mik67, Mcm5), glial (Rlbp1, Aqp4), gliosis (GFAP), cell proliferation (Btg2), and rod (Rho and Nrl) gene expression in different cell clusters;

FIGS. 4A-4H depict that Gnat1 and Rho mRNA are expressed in the ONL and OPL MG, in which FIG. 4A and FIG. 4B show the Gnat1 and Rho mRNA expression in situ hybridization in the control retinas (FIG. 4A) and the Glast-CreERT2;Sun1:GFP mouse retinas harvested at 3 weeks post CCA injection (FIG. 4B), FIGS. 4C and 4D respectively show a magnified view of the ONL region in control (FIG. 4C) and CCA (FIG. 4D) groups, FIGS. 4E and 4F respectively show a magnified view of the INL region in control (FIG. 4E) and CCA (FIG. 4F) groups, FIG. 4G shows the number of Gnat1 mRNA dots per GFP+ cell, and FIG. 4H shows the average pixel level of Rho mRNA per GFP+ cell;

FIGS. 5A-5E depict Glul mRNA level is decreased in the ONL and OPL MG, in which FIG. 5A and FIG. 5B show the Glul mRNAs in situ hybridization in the control retinas (FIG. 5A) and the Glast-CreERT2;Sun1:GFP mouse retinas (FIG. 5B), FIG. 5C shows a magnified view of the INL region in control (FIG. 5A), FIG. 5D shows a magnified view of the ONL region in CCA group (FIG. 5B), and FIG. 5E presents the average pixel level of Glul mRNAs per GFP+ cell;

FIGS. 6A-6K depict the de novo genesis of Otx2+BP-like and HuC/D+ AC-like cells from MG by CCA, in which FIG. 6A depicts the representative retinal sections of control groups, FIG. 6B presents the representative retinal sections of Glast-CreERT2; tdTomato mice at 4 months post injection of CCA, FIG. 6C depicts a magnified view of the ONL region in FIG. 6B, FIG. 6D is a magnified view around the INL region in FIG. 6B, FIG. 6E shows the quantification of tdT+EdU+Otx2+/tdT+EdU+%, FIG. 6F depicts the representative retinal sections immunostained with EdU and HuC/D of control groups, FIG. 6G depicts the representative retinal sections immunostained with EdU and HuC/D of control groups Glast-CreERT2; tdTomato mice at 4 months post injection of CCA, FIG. 6H displays a magnified image of the marked region in FIG. 6F, FIG. 6I displays a magnified image of the marked region in FIG. 6G, FIG. 6J shows the quantification of tdT+EdU+HuC/D+/tdT+EdU+%, and FIG. 6K shows that most ONL MG daughter cells retain apical processes; and

FIGS. 7A-7I shows that CCA does not lead to retinal neoplastic transformation, in which FIG. 7A shows the visual acuity of the wild type mice at 1 year after CCA injection, FIG. 7B presents the measured scotopic ERG of the wild type mice at 1 year after CCA injection, FIG. 7C displays the measured photopic ERG of the wild type mice at 1 year after CCA injection, FIG. 7D and FIG. 7E display Sox9 immunofluorescence images of the retinas obtained from untreated control mice (FIG. 7D) and the WT mice one year after CCA injection (FIG. 7E), FIG. 7F and FIG. 7G respectively are the zoom-in images of the indicated area in FIG. 7D and FIG. 7E, FIG. 7H presents the quantification of the number of Sox9+ cells in retinas at 2 weeks or one year after CCA injection and in age-corresponding WT retinas, and FIG. 7I depicts the quantification of the number of Sox9+ cells in each retinal layer

### DETAILED DESCRIPTION

In the following description, compositions, and/or methods of treating retinal degeneration and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

As used herein, the term “agonist” refers to a substance that binds to a specific receptor and activates it to produce a biological response. In pharmacology, agonists are often drugs or endogenous molecules that mimic the action of naturally occurring substances by stimulating the same receptors they target. In the context of gene therapy, an agonist can also refer to any intervention, including genetic modifications, that enhances or promotes the activity of a specific gene or its protein product, thereby triggering the desired therapeutic effect.

As used herein, the term “antagonist” refers to a substance that binds to a specific receptor but does not activate it. Instead, it blocks or dampens the biological response that would be triggered by an agonist at the same receptor. Antagonists inhibit the action of agonists or endogenous molecules by preventing them from binding to the receptor or by interfering with their action. In the context of gene therapy, an antagonist can also refer to any intervention, including genetic modifications, that reduces or inhibits the activity of a specific gene or its protein product, thereby counteracting or mitigating a biological response.

As used herein, the term “vector” refers to a recombinant plasmid or virus that comprises a nucleic acid to be delivered into a host cell, either in vitro or in vivo.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P-NH2) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

As used herein, the term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

A “recombinant AAV vector (rAAV vector)” refers to a polynucleotide vector comprising one or more heterologous sequences (i.e., nucleic acid sequence not of AAV origin) that are flanked by at least one AAV ITR. Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). A rAAV vector can be in any of a number of forms, including, but not limited to, plasmids, linear artificial chromosomes, complexed with lipids, encapsulated within liposomes, and encapsidated in a viral particle, e.g., an AAV particle. A rAAV vector can be packaged into an AAV virus capsid to generate a “recombinant adeno-associated viral particle (rAAV particle)”.

An “inverted terminal repeat” or “ITR” sequence is a term well understood in the art and refers to relatively short sequences found at the termini of viral genomes which are in opposite orientation. An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contain several shorter regions of self-complementarity (designated A, A′, B, B′, C, C′ and D regions), allowing intrastrand base-pairing to occur within this portion of the ITR.

An “rAAV virus” or “rAAV viral particle” refers to a viral particle composed of at least one AAV capsid protein and an encapsidated rAAV vector genome.

The rAAV vectors of the invention may be any AAV serotype, such as the serotype AAVrh.74, AAV1, AAV2, AAV4, AAV5, AAV6, AAV7, AAV7m8, AAV8, AAV9, AAV10, AAV11, AAV12 or AAV13.

The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome. In another aspect, it may be transcribed into a molecule that mediates RNA interference, such as miRNA, siRNA, or shRNA.

The term “genome copies” as used in reference to a viral titer, refer to the number of virions containing the recombinant AAV DNA genome, regardless of infectivity or functionality.

The retinal degenerative diseases include retinitis pigmentosa, age-related macular degeneration, and diabetic retinopathy.

In accordance with a first aspect of the present invention, a method of treating retinal degenerative diseases in a subject who is in need of such treatment is provided. This method involves the strategic administration of specific agents to Müller glia (MG) cells within the retina to induce their proliferation and partial differentiation into neuron-like cells, thus addressing the cellular deficits characteristic of retinal degeneration.

In the method, an agonist is administered to selectively elevate the expression of cyclin D1 in MG cells. Cyclin D1 is a critical regulator of cell cycle progression, and its elevated expression in MG cells promotes their re-entry into the cell cycle, thereby stimulating their proliferation. This proliferation is a crucial step in generating new cells that can potentially replace the degenerated retinal neurons.

Concurrently, an antagonist is administered to selectively knock down the expression of p27^{kip1} in MG cells. p27^{kip1} is a known inhibitor of the cell cycle, and its downregulation is essential to remove the inhibitory constraints on cell cycle progression, further promoting the proliferation of MG cells. By knocking down p27^{kip1}, the method ensures that the proliferative signals mediated by elevated cyclin D1 expression are not counteracted, thereby maximizing the proliferative response of the MG cells.

For effective delivery, the administration of the agonist and the antagonist is designed to enable their delivery to the subject's subretinal space or vitreous. These are the anatomical regions where MG cells reside, and delivering the agents directly to these areas ensures that the therapeutic agents reach their target cells in adequate concentrations to exert their biological effects.

The synergistic effect of cyclin D1 overexpression and p27kip1 knockdown not only drives MG proliferation but also induces partial differentiation of these proliferated MG cells towards neuron-like cells. This partial differentiation is essential for the MG cells to acquire characteristics and functionalities of retinal neurons, which are necessary to replace the lost or damaged neurons in retinal degenerative diseases.

The targeted retinal degenerative diseases include diabetic retinopathy, age-related macular degeneration (AMD), retinitis pigmentosa, Stargardt's disease, macular hole, and bright light-induced retina damage.

In summary, the method described leverages the dual strategy of promoting cell cycle entry and removing cell cycle inhibition in MG cells, facilitating their proliferation and partial differentiation. It provides a therapeutic strategy for treating retinal degenerative diseases by harnessing the regenerative potential of MG cells within the retina.

In accordance with a second aspect of the present invention, a composition for treating retinal degenerative diseases in a subject in need thereof is presented. This composition includes an agonist that selectively elevates the expression of cyclin D1 in MG cells, an antagonist that selectively knocks down the expression of p27^{kip1} in MG cells, and a pharmaceutically acceptable addition.

In some embodiments, the agonist and antagonist are integrated into a gene therapy technique involving rAAV, a recombinant adenovirus, a recombinant lentivirus, a recombinant retrovirus, a recombinant vaccinia virus, a recombinant herpes simplex virus, a naked plasmid, a lipid nanoparticle, a peptide-based vector and a polymer-based vector.

In one embodiment, the agonist and antagonist are integrated into a gene therapy technique involving a rAAV vector for delivering gene therapy to the retinal region. The rAAV vector includes a promoter DNA sequence obtained from the human glial fibrillary acidic protein (GFAP) gene, a DNA sequence encoding cyclin D1, and a short hairpin RNA sequence targeting the p27kip1 gene. The short hairpin RNA (shRNA) sequence is encoded in the 3′ untranslated region (UTR) of the cyclin D1 gene. This configuration ensures the precise delivery and expression of the therapeutic agents at the target site.

In some embodiments, the promoter DNA sequence is, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, 99%, or 100% sequence identity to SEQ ID NO: 01.

Cyclin D1 plays a significant role in the transition from the G1 phase to the S phase of the cell cycle, thereby promoting cell proliferation. The inclusion of the cyclin D1 gene in the vector is designed to stimulate the proliferation of MG, a necessary step for initiating retinal regeneration. In preferred embodiments, the DNA sequence encoding cyclin D1 is at least 85% identical to the DNA sequence of SEQ ID NO: 02, ensuring the functional expression of cyclin D1 protein.

For example, the DNA sequence encodes a functional cyclin D1 protein, wherein the sequence is, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, 99%, or 100% sequence identity to SEQ ID NO: 02, wherein the protein retains cyclin D1 activity.

The shRNA sequence in the rAAV specifically targets the p27^{kip1} gene. The p27^{kip1} protein is a known inhibitor of cell cycle progression, and its downregulation is essential for enabling cell proliferation. The shRNA sequence designed to target p27^{kip1} is strategically placed in the 3′ untranslated region (UTR) of the cyclin D1 gene. This configuration allows for the concurrent expression of cyclin D1 and the knockdown of p27kip1, maximizing the proliferative response in MG cells. In preferred embodiments, the shRNA sequence is at least 85% identical to the DNA sequence of SEQ ID NO: 03. In some embodiments, the shRNA sequence is, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, 99%, or 100% sequence identity to SEQ ID NO: 03.

The rAAV vector is designed to stimulate MG cells to overexpress cyclin D1 and knock down the expression of p27^{kip1}. This dual action promotes the proliferation of MG cells and their partial differentiation towards neuron-like cells, addressing the cellular deficits associated with retinal degenerative diseases.

The composition includes pharmaceutically acceptable additions, which may comprise one or more of an excipient, a stability additive, a carrier, a diluent, or a solubilizer. These additions ensure the stability, solubility, and proper delivery of the rAAV vector, enhancing its therapeutic efficacy. The formulation of the composition is designed to enable effective delivery to the subject's subretinal space, either through the cornea or the blood-retinal barrier, ensuring that the therapeutic agents reach the target site within the retina.

In terms of administration forms, the composition can be adapted to either an immediate-release form or a controlled-release form, depending on the therapeutic requirements. The delivery of the composition can be achieved through various approaches, including intravitreal injection, subretinal injection, or suprachoroidal injection. These methods are chosen based on their ability to deliver the composition precisely to the retinal tissues.

“Intravitreal injection”, a common method involves injecting drugs directly into the vitreous humor of the eye.

“Subretinal injection”, a surgical procedure involves injecting drugs directly into the subretinal space. Briefly, the vitreous gel is taken out and a bleb is raised underneath the retina, peripheral to the central retina, by injecting the composition.

Drug formulation and delivery system are crucial for ophthalmic applications. The delivery of the composition is necessary to enables delivery to the subject's subretinal space or vitreous. The delivery technologies encompass the following characteristics: (1) easy and noninvasive administration; (2) an efficient delivery system; (3) compatible with ocular tissues; (4) target-specific for the indicated ocular diseases; and (5) a controlled-release system, which keeps active agents for a prolonged period (several months to several years) in the retina.

Sustained-release formulations can be administered once daily or even less frequently. Sustained-release formulations can be based on matrix technology.

A pulsed-release dosage form includes an immediate-release dosage form including the composition; and a delayed-release dosage form including the composition.

In one embodiment, a delayed-release dosage form can be combined with an immediate-release dosage form to provide a pulsed-release dosage form. The delayed-release dosage form may be in the form of a core which optionally includes absorption enhancers and/or water swellable substances. Pulsed-release dosage forms allow for control of the plasma levels of the composition.

The term of “immediate-release” means that a conventional or non-modified release form in which greater than or equal to about 75% of the active agent is released within two hours of administration, preferably within one hour of administration.

The term “controlled-release” is a dosage form in which the release of the active agent is controlled or modified over a period of time. Controlled can mean, for example, sustained, delayed or pulsed-release at a particular time. Alternatively, controlled can mean that the release of the active agent is extended for longer than it would be in an immediate-release dosage form, i.e., at least over several hours, such as greater than four hours, preferably greater than eight hours.

The term “sustained-release” or “extended-release” is meant to include the release of the active agent at such a rate that blood (e.g., plasma) levels are maintained within a therapeutic range but below toxic levels for at least about 8 hours, preferably at least about 12 hours after administration at steady-state. The term “steady-state” means that a plasma level for a given active agent has been achieved and which is maintained with subsequent doses of the drug at a level which is at or above the minimum effective therapeutic level and is below the minimum toxic plasma level for a given active agent.

The term “delayed-release” means that there is a time-delay before significant plasma levels of the active agent are achieved. A delayed-release formulation of the active agent can avoid an initial burst of the active agent, or can be formulated so that release of the active agent in eye ball or muscle layer is avoided and absorption takes places in retina.

A “pulsed-release” formulation can contain a combination of immediate-release, sustained-release, and/or delayed-release formulations in the same dosage form. A “semi-delayed-release” formulation is a pulsed-released formulation in which a moderate dosage is provided immediately after administration and a further dosage some hours after administration.

Moreover, the composition can be formulated into various administration forms such as injections, hydrogels, ultrasonic ocular drug delivery systems, drug-eluting implants, nanoparticle-mediated delivery systems, and intravitreal microneedles. These diverse forms provide flexibility in administration and cater to different patient needs and clinical scenarios.

“Drug-eluting implants”, such as sustained-release devices or drug-eluting micro/nanoparticles, can be surgically placed in the eye. These devices release drugs over an extended period and can be designed for targeted subretinal delivery.

“Nanoparticle-mediated delivery”, nanoparticles are engineered to encapsulate drugs and improve their bioavailability. Surface modifications can be made to enhance their affinity for the retina and facilitate subretinal delivery. In one embodiment, the composition is delivered by a nanoparticle or microemulsion drug delivery system, which enhances solubility and improve delivery efficiency by surface-conjugating active targeting agonists or improves drug solubilization capacity and bioavailability. Moreover, the systems also reduce drug toxicity, prolong the residence time, and protect biological drugs from degradation. Another direction is the integration of nanotechnology with other delivery systems, such as ultrasonic ocular drug delivery systems, drug-loaded contact lenses, and hydrogel.

“Ultrasound-mediated delivery”, uses ultrasound to enhance drug penetration into the retina. The application of ultrasound waves can transiently disrupt barriers, facilitating drug entry into the subretinal space.

“Electroporation”, involves applying electrical pulses to cells, temporarily increasing cell membrane permeability. This technique has been used for enhancing the penetration of drugs into the retina, including the subretinal space.

“Intravitreal microneedles”, are designed to penetrate the retina and deliver drugs to specific layers, including the subretinal space. This is a minimally invasive approach compared to traditional subretinal injections.

In addition to the rAAV vector, the composition can be delivered in combination with other neurogenic factors to enhance retinal regeneration. This combined approach aims to maximize the regenerative potential of Müller glia by not only stimulating their proliferation but also promoting their differentiation into various retinal cell types, thereby addressing the multifaceted nature of retinal degenerative diseases.

Overall, this detailed description outlines the composition's components, formulation strategies, and potential administration methods, highlighting its therapeutic potential for treating retinal degenerative diseases by leveraging the regenerative capacity of MG.

In accordance with a third aspect of the present invention, a usage of the aforementioned composition for treating retinal degenerative diseases is provided. The usage includes administering an amount effective of the composition to the subject in need thereof.

In some embodiments, the retinal degenerative diseases include diabetic retinopathy, AMD, retinitis pigmentosa, Stargardt's disease, macular hole, and bright light-induced retina damage.

### EXAMPLES

### Immunohistochemistry

Retinae are dissected and fixed in 4% formaldehyde in PBS for 30 min at room temperature and sectioned at 20-am thickness by cryostat. Retinal sections are blocked in 5% BSA in PBST (PBS with 0.1% Triton X-100), stained with primary antibodies at 4° C. overnight, and washed three times with PBST. Primary antibodies used in the present invention include rabbit anti-p27kip1 (1:200, PA5-16717; Thermofisher); rabbit anti-Cyclin D1 antibody (1:300, 26939-1-AP; Proteintech), and rabbit anti-Sox9 antibody (1:1000, AB5535; Millipore). Sections are stained using secondary antibodies (Jackson ImmunoResearch) for two hours at room temperature. Cell nuclei are counterstained with DAPI (Sigma).

### Western Blot Analyses

Retinae are homogenized in lysis buffer (10 mM Tris-HCl [pH 8.0], 140 mM NaCl, 1 mM EDTA, 0.5 mM EGTA, 1% Triton X-100, 0.1% Sodium Deoxycholate, 0.1% SDS,) containing protease inhibitor cocktail (Sigma) at 4° C. Clarified protein lysates are quantified by the BCA assay and 10 to 25 ag of protein is loaded for western-blot analysis. Primary antibodies rabbit anti-p27kip1 (1:1000, PA5-16717; Thermofisher), rabbit anti-Cyclin D1 (1:2000, 26939-1-AP; Proteintech), and mouse anti-α-tubulin (1:1000, ab7291; Abcam) were incubated overnight at 4° C. HRP conjugated secondary antibodies are incubated for 1 hour at room temperature. Protein detection is performed using an enhanced chemiluminescence kit (Bio-Rad). Quantification is done using Fiji software.

### EdU Incorporation and BrdU Detection Assay

5′-ethynyl-2′-deoxyuridine (EdU, 50 mg/kg, Abeam ab146186) or 5-bromo-2′ deoxyuridine (BrdU, 100 mg/kg, Abcam 142567) is intraperitoneally injected to label the cells in the S phase. EdU staining is performed using the Click-iT™ EdU Alexa Fluor™ 488 Imaging Kit (C10337). For BrdU detection, the retinal sections are incubated with 2 M HCl for 1 hour at room temperature. The sections are rinsed with PBST and incubated with a blocking buffer containing 5% BSA in PBST for 2 hours at room temperature. Primary antibody mouse anti-BrdU (1:300, ab8152; Abcam) is stained overnight at 4° C., and secondary antibodies (Jackson ImmunoResearch) are stained for 2 hours at room temperature.

scRNA-Seq

### Library Preparation and Sequencing of Single Cells

The FACS-sorted cells from each sample are filtered through 40 μm strainer (pluriStrainer) and processed with Chromium Next GEM Single Cell 3′ GEM, Library & Gel Bead Kit v3.1 (1000269), Chromium Next GEM Chip G Single Cell Kit (1000127) and Chromium Controller (10× Genomics) according to the manufacturer's protocol. The constructed libraries are sent to Novogene (Beijing) for NovaSeq paired-end 150 bp sequencing and produced 500 Gb of raw data.

Preprocessing, Filtering and Clustering of scRNA Data

Sequencing results are preprocessed with 10× Genomics Cell Ranger 6.1.1. (Zheng et al., 2017) for demultiplexing, barcode assignment, and unique molecular identifier (UMI) quantification using a standard pipeline for 10× for each of the three samples. Indexed mouse genome mm10 with added tdTomato and GFP transgenes are utilized as a reference. Reads mapping to introns cover more than 34% of all reads. Therefore, they are taken into account in feature counts by using ‘include_introns’ option in cellranger count step of the pipeline. Cell Ranger aggregation is applied for merging runs from three samples.

Quality control, filtering, dimensional reduction, and clustering of the data are carried out using the Seurat package in R (Stuart et al., 2019). The cells with less than 2,000 expressed features, total UMI count higher than 100,000 and percentage of mitochondrial transcripts more than 15% are disregarded. Features of the Y chromosome, as well as Xist and Tsix genes of the X chromosome, are excluded in order to avoid confusing effects of gender.

Further filtering of cells is carried out by only including cells expressing tdTomato. Potential cell duplicates are estimated using DoubletFinder method (McGinnis et al., 2019) implemented in R (with parameter settings PC=10, pK=0.28, pN=0.3). The threshold for the expected doublet formation rate is set to 10% to reflect the assumption that cell duplicates can appear relatively commonly in the MG cells. Detected duplicates are excluded from the data.

For the remaining cells, Uniform Manifold Approximation (UMAP) dimension reduction based on 8 principal components (PCs) is applied, and cells are clustered using the graphical clustering method in Seurat. Cell types are identified using known marker genes.

Analysis of scRNA-Seg Data

The cell cycle state of each cell is defined by the Cell Cycle Scoring function in the Seurat R package. The cells with G2M and S scores lower than 0.1 are defined to be proliferating. Differential gene expression analysis between different cell types is performed using Deseq2. Enrichment analysis of top 200 up- and downregulated genes is performed for Gene Ontologies (GO), KEGG and Reactome pathways using Gprofiler2 R interface to g:Profiler.

### Example 1. Simultaneous p27^{Kip1} Downregulation and Cyclin D1 Overexpression Drive Robust MG Proliferation in the Uninjured Mouse Retina

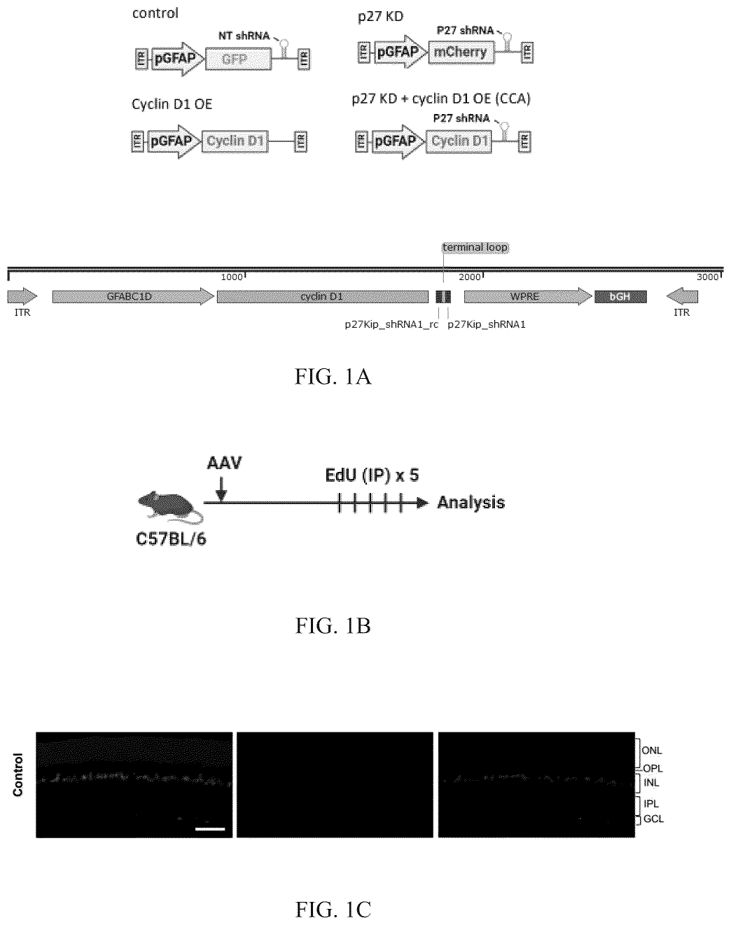

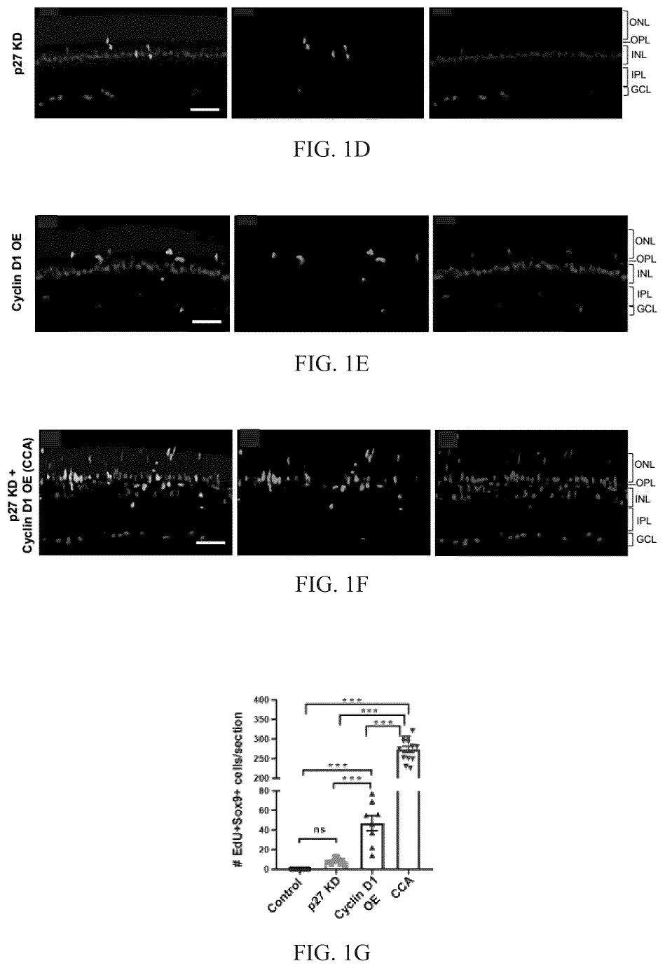

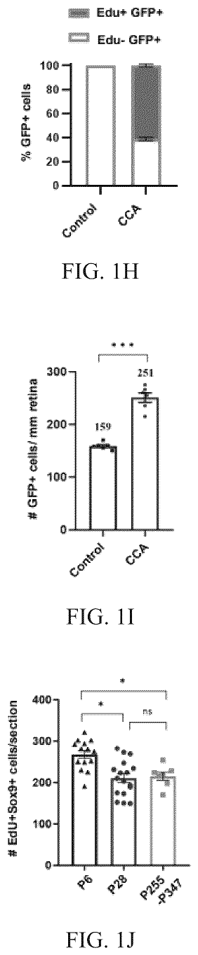

To test the hypothesis that adult mouse MG are maintained in a quiescent state by high levels of p27^{Kip1} and low levels of cyclin D1, it is investigated that whether spontaneous MG proliferation can be induced by directly altering the levels of these two key cell cycle regulators. In this embodiment, AAV₇ₘ₈ vectors and a promoter sequence cloned from the glial fibrillary acidic protein (GFAP) gene are utilized to drive MG-specific gene expression (FIG. 1A). AAV₇ₘ₈ vectors are injected intravitreally into the eyes of C57BL/6 pups at postnatal day 6 (P6), followed by intraperitoneal injections of 5-ethynyl-2′-deoxyuridine (EdU) to label MG that have entered the S phase of the cell cycle (FIG. 1B). The intravitreal injection is performed using a pulled angled glass pipette controlled by a FemtoJet (Eppendorf). The tip of the needle is passed through the sclera at the equator, near the dorsal limbus of the eyeball, and entered into the vitreous cavity. The injection volume of AAVs is 0.5 μl per eye for P6 injection and 1 per eye for adult injection. In the control retinas infected with AAV₇ₘ₈-GFAP-GFP-nontarget (NT) short hairpin RNA (shRNA) virus, all MG, identified by Sox9 expression aligning in the inner nuclear layer (INL), are negative for EdU labeling (FIG. 1C).

As shown in FIG. 1A, pAAV-GFAP-GFP vector plasmid is cloned by replacing the CMV promoter with a 684 bp ABC1D region of the mouse GFAP gene promoter into the pAAV-CMV-GFP vector. cDNAs encoding mouse cyclin D1 are cloned into AAV plasmids by Gibson ligation. AAV-shRNA vectors are cloned by replacing the GFP sequence with mCherry-shRNA in the pAAV-GFAP-GFP vector. pAAV-GFAP-cyclinD1-P27shRNA and pAAV-GFAP-GFP-LacZshRNA are cloned by replacing mCherry sequence with cyclinD1 and GFP from the pAAV-GFAP-mCherry-P27shRNA1 and pAAV-GFAP-mCherry-LacZshRNA, respectively. pAAV rep/Cap 2/2, and Adenovirus helper plasmids are obtained from the University of Pennsylvania Vector Core, Philadelphia. pAAV7m8 plasmid (#64839) is purchased from addgene. The obtained AAV-GFAP-GFP, AAV-GFAP-mCherry-p27shRNA1-WPRE and AAV-GFAP-mCherry-LacZshRNA-WPRE have the sequences as listed in SEQ ID NO: 04, SEQ ID NO: 05, and SEQ ID NO: 06, respectively.

AAV is produced in HEK293T cells (HCL4517; Thermo Scientific) by AAV vector, rep/cap packaging plasmid, and adenoviral helper plasmid co-transfection followed by iodixanol gradient ultracentrifugation. Purified AAVs are concentrated with Amicon 100K columns (EMD Millipore) to a final titer of 2-4×10¹³ genome copies/mL. Protein gels are run to determine virus titers.

When the retina is infected with AAV₇ₘ₈-GFAP-mCherry-p27^{Kip1} shRNA, which expresses a highly efficient p27^{Kip1} shRNA, a small number of MG cells re-entered the cell cycle (FIG. 1D and FIG. 1G). However, these proliferating MG cells account for less than 1% of the total MG population. Similarly, overexpressing cyclin D1 alone through AAV₇ₘ₈-GFAP-CyclinD1 infection stimulates a subset of MG cells to proliferate, resulting in a three-fold increase in the number of EdU+MG cells compared to p27^{Kip1} knockdown (FIG. 1E and FIG. 1G).

Finally, the AAV₇ₘ₈-GFAP-CyclinD1-p27^{Kip1} shRNA vector, which simultaneously overexpresses cyclin D1 and suppressed p27^{Kip1}, has the most significant impact on MG proliferation, with a five-fold increase in EdU+Sox9+ cells compared to cyclin D1 overexpression alone (FIG. 1F and FIG. 1G). These findings suggest that the combination of p27^{Kip1} knockdown and cyclin D1 overexpression, referred to hereinafter as the cell cycle activator (CCA), synergistically promotes MG proliferation. This highlights the critical importance of both low levels of p27^{Kip1} and high levels of cyclin D1 in unlocking the mitogenic capacity of MG cells.

In some embodiments, the CCA vector, 768-AAV-GFABC1D-cyclinD1-p27-shRNA1-WPRE-bGH, has a sequence as listed in SEQ ID NO: 07.

A transgenic mouse line, Glast-CreERT2; SUN1, where MG nuclei are labeled by nuclear membrane-bound GFP, is used to quantify the percentage of MG that re-enter the cell cycle. In the area with the most efficient virus infection, approximately 45% of SUN1:GFP+MG are EdU positive (FIG. 1H), and the total number of MG increases by about 50% (FIG. 1I), indicating that nearly half of the MG cells re-enter the cell cycle. Previous research shows that the ability of retinal regeneration by various stimuli declines with the age of the mice. The efficiency of CCA in driving MG proliferation in neonatal (P6), young (P28), and aged (>8 months) mouse retinas is examined. Remarkably, MG proliferation induced by CCA remains robust in young adult and aged mice, with more than 200 EdU+Sox9+ cells per section (FIG. 1J). These findings suggest that CCA is robust in driving MG proliferation in both young and aged animals.

### Example 2. MG Proliferation Driven by CCA is Self-Limiting

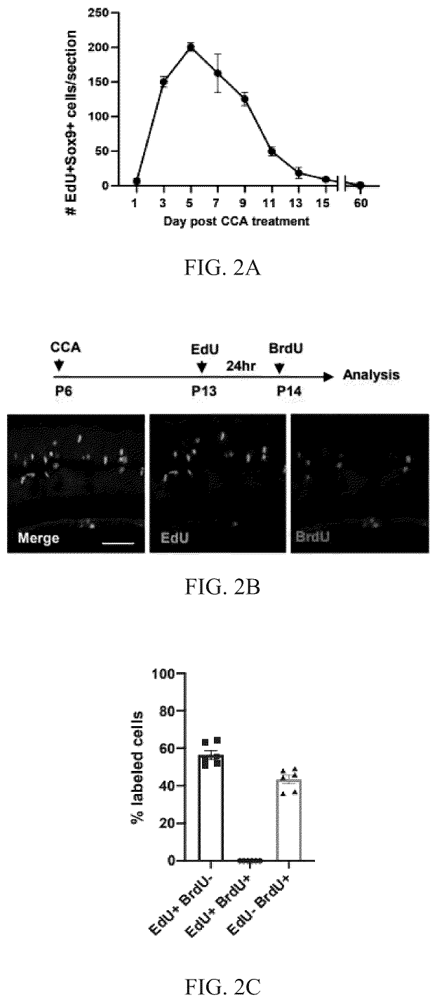

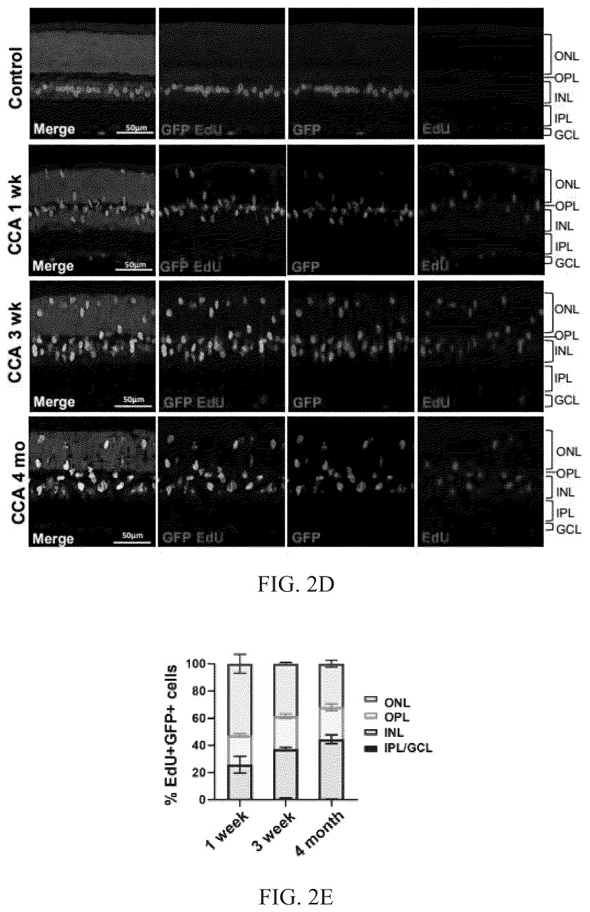

With the concern that p27^{Kip1} suppression and cyclin D1 overexpression may lead to uncontrolled cell proliferation and retinal tumorigenesis, the proliferative capacity of MG driven by CCA is further analyzed. Firstly, the duration of MG proliferation after CCA treatment is examined using a time-course EdU incorporation assay. On various days post-CCA injection, EdU is administered to label the cells undergoing proliferation. As shown in FIG. 2A, the results reveal that MG proliferation starts as early as the third day after CCA injection, peaks around the fifth day, and then gradually decreases. By two weeks post-CCA injection, only a few MG are observed to have re-entered the cell cycle, and by two months post-CCA injection, MG proliferation has mostly ceased. These findings suggest that MG proliferation is largely completed within two weeks post-CCA treatment, likely due to the dilution of AAV episomal genome copies in the dividing cells. Additionally, an EdU/BrdU double-labeling assay is performed to examine whether MG undergo one or multiple cell divisions after CCA treatment. A single injection of EdU is given at seven days post-CCA treatment, followed by BrdU injection 24 hours later (FIG. 2B). Retinas are collected two days after BrdU injection to evaluate if any MG continuously enter the S phase of the cell cycle. While there are numerous EdU+ cells and BrdU+ cells, no cells are co-labeled with EdU and BrdU (FIG. 2B and FIG. 2C), indicating that MG undergo only one cell division subsequent to CCA treatment.

Next, the distribution of proliferated MG in the retina over time is further examined. Due to variations in the labeling efficiencies of MG by Tamoxifen-induced reporter expression and EdU among experiments and individual animals, the percentages of EdU+GFP+MG in each retinal layer are utilized to assess the distribution of proliferated MG. During retina development, proliferating retinal progenitor cells migrate in an apical-basal manner, whereas post-mitotic cells migrate basally to reach their final laminar position. The proliferating MG undergo similar interkinetic migration towards the apical surface of the retina, as the majority of EdU+GFP+MG nuclei are observed in the outer nuclear layer (ONL) and outer plexiform layer (OPL) (˜53% and ˜22%, respectively) one week after CCA treatment, which is around the peak time of MG proliferation (FIG. 2D and FIG. 2E). To further explore whether the ONL MG daughter cells eventually return to the INL over an extended period, the distribution of EdU+MG cells at three weeks and four months post-CCA treatment is also evaluated. It is found that while the percentage of INL EdU+MG increases slightly over time, about half of EdU+MG still remain in the ONL and OPL (FIG. 2D and FIG. 2E). Across the three time points examined, the absence of EdU+GFP+MG in the inner plexiform layer (IPL) and ganglion cell layer (GCL) suggests that migration of MG daughter cells to the GCL does not occur, at least without injury (FIG. 2D and FIG. 2E). These findings lead us to investigate whether the long-term location of MG daughter cells in the ONL and OPL is associated with changes in cell fate.

### Example 3. Cell Cycle Re-Activation Drives Downregulation of IFN-γ Pathway and MG Dedifferentiation

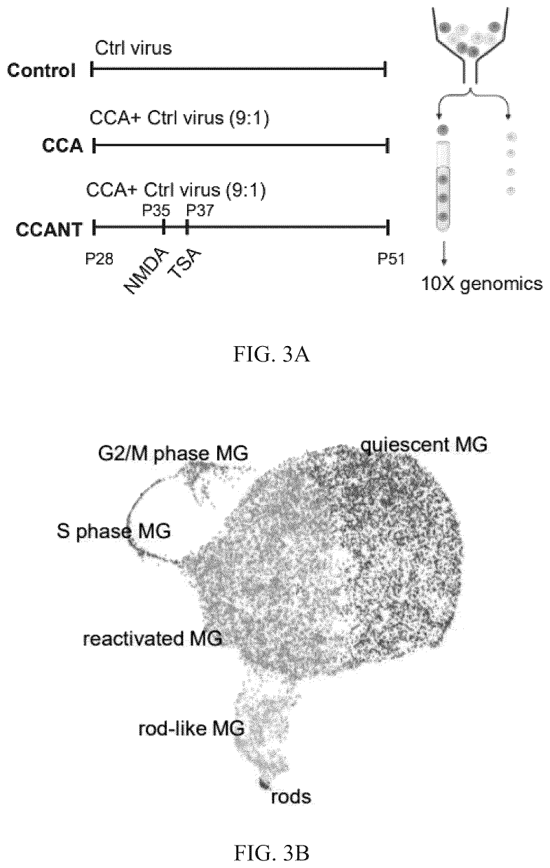

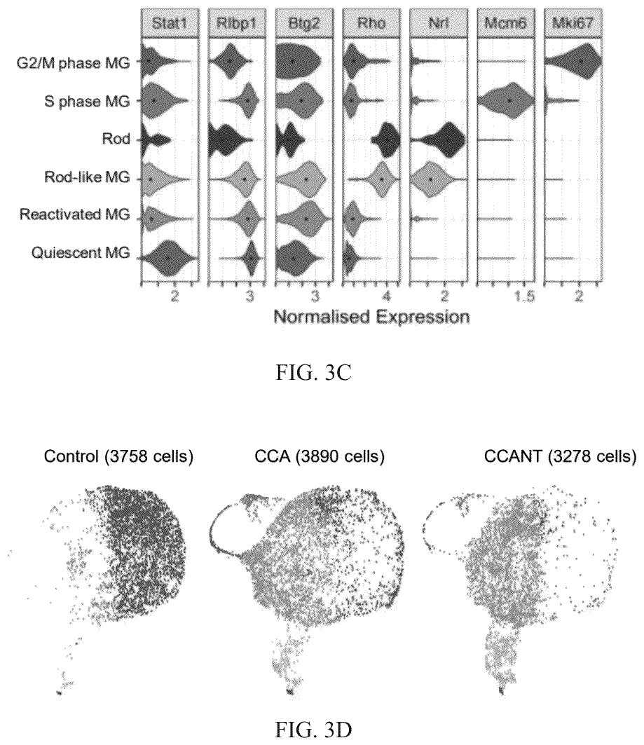

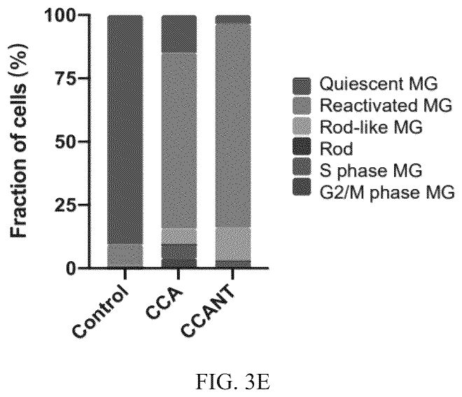

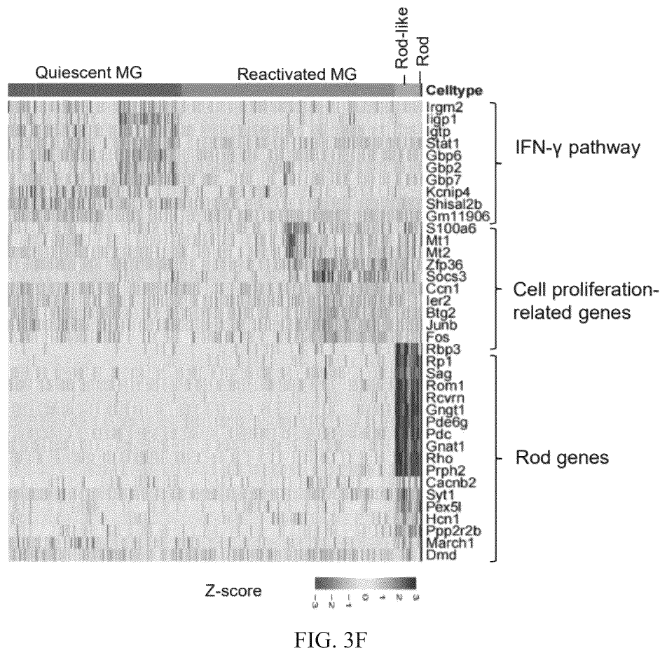

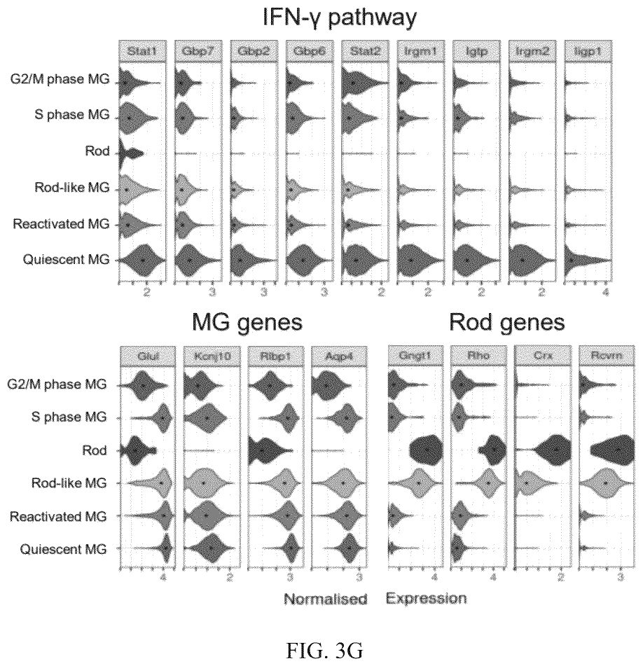

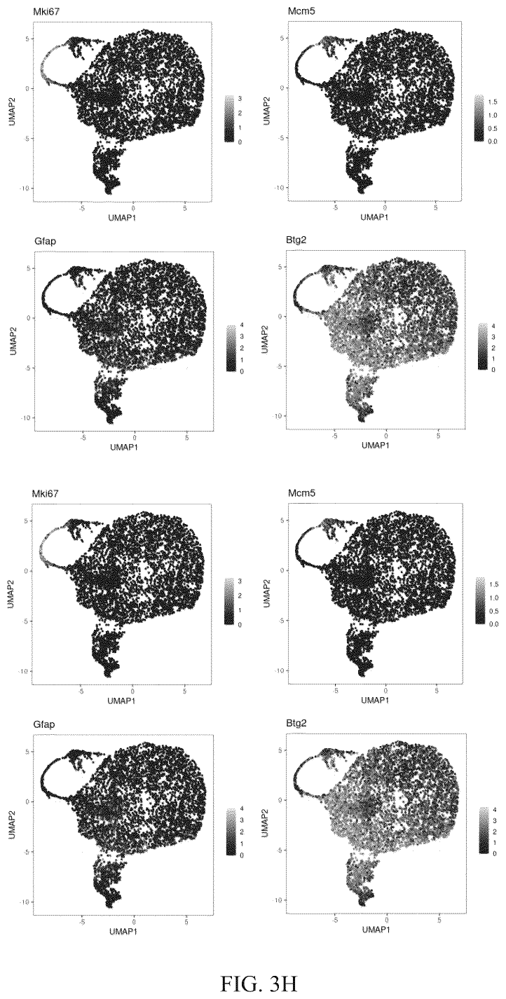

To unbiasedly assess the cell fate of MG and their daughter cells, single-cell RNA-Seq (scRNA-Seq) analysis is performed on mouse retinas that receive CCA treatment. AAV₇ₘ₈-GFAP-cyclin D1-p27^{Kip1} shRNA and AAV₇ₘ₈-GFAP-GFP-NT shRNA (9:1 mixed) are co-injected to stimulate MG proliferation in 4-week-old Glast-Cre^{ERT2};tdTomato mice (FIG. 3A). Additionally, 10% AAV₇ₘ₈-GFAP-GFP-LacZsh is added to serve as an indicator of infection success. The control group is injected with AAV₇ₘ₈-GFAP-GFP-NT shRNA to account for any non-specific effects caused by virus injection and/or shRNA expression. Three weeks after virus injection, retinas with good infection are collected, and tdTomato+ cells are isolated by fluorescence-activated cell sorting (FACS), followed by scRNA-Seq using the 10× Genomics platform (FIG. 3A). As previous studies have demonstrated that NMDA-induced retinal damage and the histone deacetylase inhibitor Trichostatin A (TSA) improve MG proliferation and reprogramming, a group of CCA-treated mice also receives NMDA on day 7 and TSA on day 9 post-CCA injection (CCA+NMDA+TSA, referred to as CCANT) to maximize the potential reprogramming effect of CCA.

After computational processing to exclude tdTomato-cells and doublets, 3,758 cells are profiled in the control group, 3,890 cells in the CCA group, and 3,278 cells in the CCANT group. Principal component analysis (PCA) separates the cells into six different clusters (FIG. 3B), which are quiescent MG, reactivated MG, MG in G2/M phase, MG in S phase, rod-like MG, and rods, as annotated by known retinal cell type markers (FIG. 3C). In the control group, the vast majority of MG (>90%) remain quiescent, expressing high levels of MG genes such as Rlbp1 and Aqp4 (FIGS. 3C-3H). In the CCA group, a small percentage of cells express the G2/M phase gene Mki67 and S phase gene Mcm5, indicating a small number of proliferating MG at 3 weeks post-CCA treatment (FIG. 3H). In this group, the majority of MG (˜70%) form a separate cluster, referred to as reactivated MG. In this cluster, there is downregulation of MG genes (such as Rlbp1 and Aqp4) and upregulation of reactive gliosis gene Gfap and cell cycle inhibitor Btg2 (FIG. 3H).

Interestingly, the top differentially expressed genes (DEGs) between reactivated MG and quiescent MG are IFN-γ pathway genes, including Stat1, Stat2, Irgp1, Irgm1, and Igtp, which are downregulated in reactivated MG (FIGS. 2F-2H). However, Stat3 is not downregulated as much as Stat1 or Stat2. STAT activation has been shown to reduce ASCL1-induced retinal regeneration. STAT1 and STAT2 primarily mediate the IFN-γ pathway, associated with inflammation and known as antitumor cytokine signaling that facilitates immunosurveillance in tumor cells. An activated IFN-γ pathway has been shown to hamper regeneration in the mouse retina. The downregulation of the IFN-γ pathway in reactivated MG may be a direct response to cyclin D1 overexpression and/or p27Kip1 knockdown. A small percentage of MG (8%) in the control virus-injected group also exhibit reactivation, likely due to retinal injury caused by virus injection and the inflammatory response to the AAV virus (FIG. 3D, E). The cell clusters of the CCANT group largely overlap with the CCA group, with CCANT having fewer proliferating cells and more reactivated MG.

### Example 3. CCA Promotes Rod Gene Expression in a Subset of MG

The rods and rod-like MG appear as two unexpected clusters in the scRNA-seq analysis. Close examination of the retina of Glast-Cre^{ERT2}; R26-tdTomato mice without Tamoxifen induction reveals that very few rods (14 Tdt+ rods in 128 whole retinal sections of 8 retina samples screened) are mislabeled by leaky tdTomato expression in a Cre-independent manner. The small rod clusters likely represent true rods mislabeled by tdTomato, which is present in all three groups in similar proportions. The rod-like MG cluster resides between the reactivated MG cluster and the rod cluster and is significantly enriched in the CCA and CCANT groups. The rod-like MG express most rod genes, including Rho, Gnat1, Rcvrn, Crx, and Nrl (FIG. 3G and FIG. 3H,), although the expression levels of rod-specific genes are lower than those of natural rods.

### Example 4. Rod-Like MG in the ONL Expresses Both Rod and MG Genes

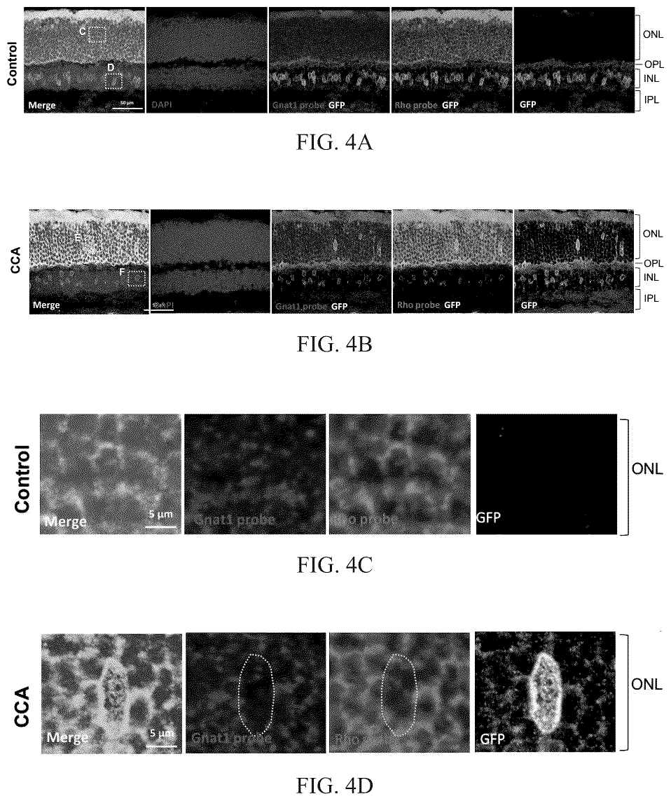

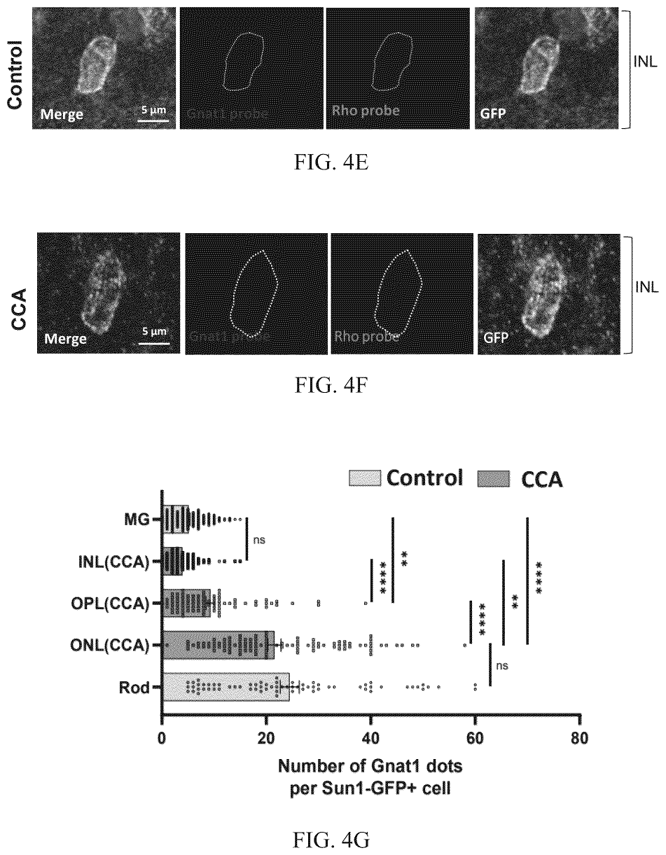

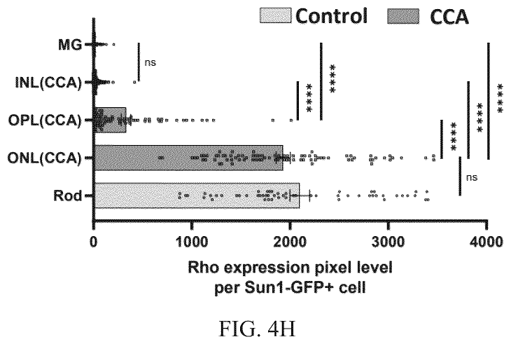

To exclude the possibility that the rod-like MG are fused MG with rod cell debris during retina isolation for scRNA-seq analysis, the expression of rod genes in the rod-like MG on retinal sections of the CCA-treated Glast-Cre^{ERT2};SUN1:GFP mice is examined. Immunoreactivity for rod proteins such as Rhodopsin and Gnat1 is present in rod outer segments (ROS), which are densely packed and distal from rod nuclei. Therefore, RNA in situ hybridization on retinal sections using the RNAscope assay is performed to analyze target RNA abundance in rod nuclei while preserving spatial information. The mRNA levels of the Rho and Gnat1 genes are examined at 3 weeks post-CCA treatment, matching the harvest time point for scRNA-seq analysis. The signals of both probes are specific as they are detected only in the ONL layer (FIG. 4A and FIG. 4B).

In control retinas, the mRNA of the two rod genes is abundantly present in the rods in the ONL (FIG. 4A and FIG. 4C), while minimal Gnat1 and Rho mRNA is detected in the MG in the INL (FIG. 4A and FIG. 4E). In the CCA-treated retina, the MG in the ONL shows clear signals of both Gnat1 and Rho mRNAs (FIG. 4B and FIG. 4D), while the INL MG maintains very low levels of Gnat1 and Rho mRNA (FIG. 4B and FIG. 4F). The number of Gnat1 fluorescent dots, serving as a reliable indicator of Gnat1 mRNA levels, was counted. The results show a significant upregulation of Gnat1 mRNA levels in the MG-derived cells in the ONL, comparable to natural rods (FIG. 4G). The MG in the OPL have intermediate levels of Gnat1 mRNA, while the MG in the INL are similar to the untreated MG controls (FIG. 4G). The Rho mRNA levels are quantified by measuring the pixel intensity due to the fusion of numerous Rho fluorescent dots, showing a similar trend of Rho mRNA expression as that of Gnat1 (FIG. 4H).

The RNAscope results demonstrate that the MG-derived cells in the ONL express high levels of rod genes, those in the OPL express intermediate levels of rod genes, and all MG in the INL retain no/low rod gene expression. This suggests that the expression level of rod genes in the MG daughter cells is associated with their position within the retinal layers.

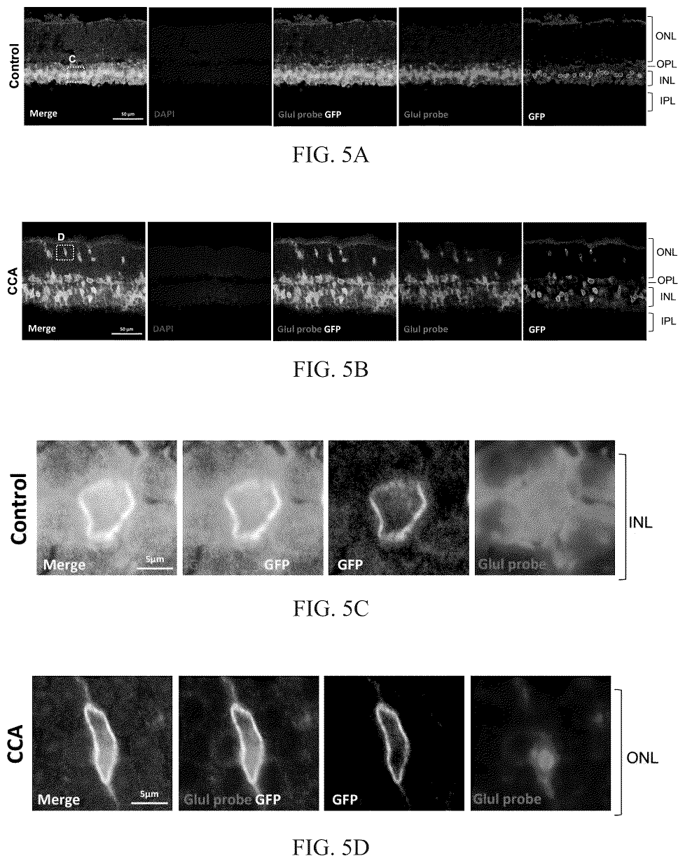

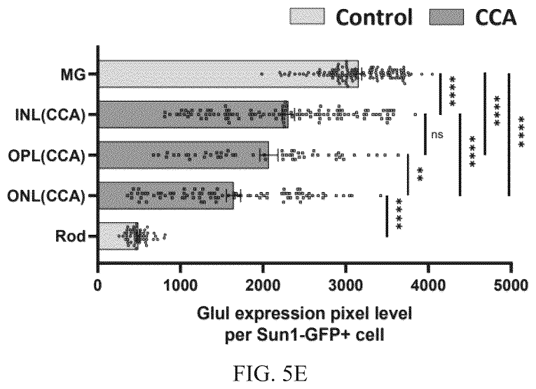

The expression level of the MG marker gene Glul, encoding Glutamate synthetase, is further examined in MG cells located in different retinal layers (FIGS. 5A-5D). After CCA treatment, MG in the INL continue to express Glul, albeit at decreased levels (FIG. 5E), consistent with the downregulation of MG genes observed in the majority of MG by scRNA-seq. MG-derived cells in the ONL express lower levels of Glul, suggesting they retain their MG identity. The RNAscope results are in alignment with the scRNA-seq analysis, indicating that MG-derived cells in the ONL express both rod and MG genes at 3 weeks post CCA treatment.

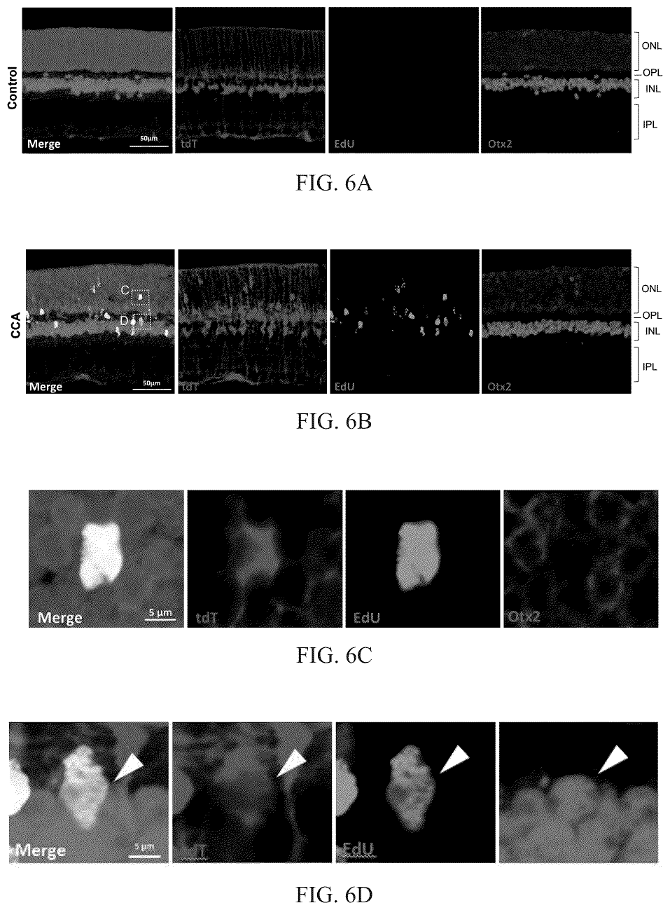

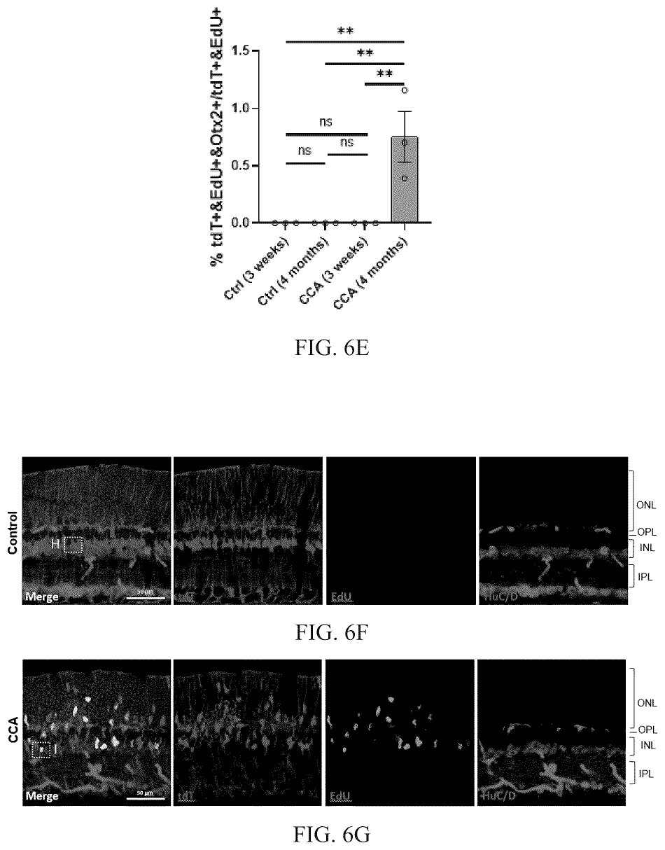

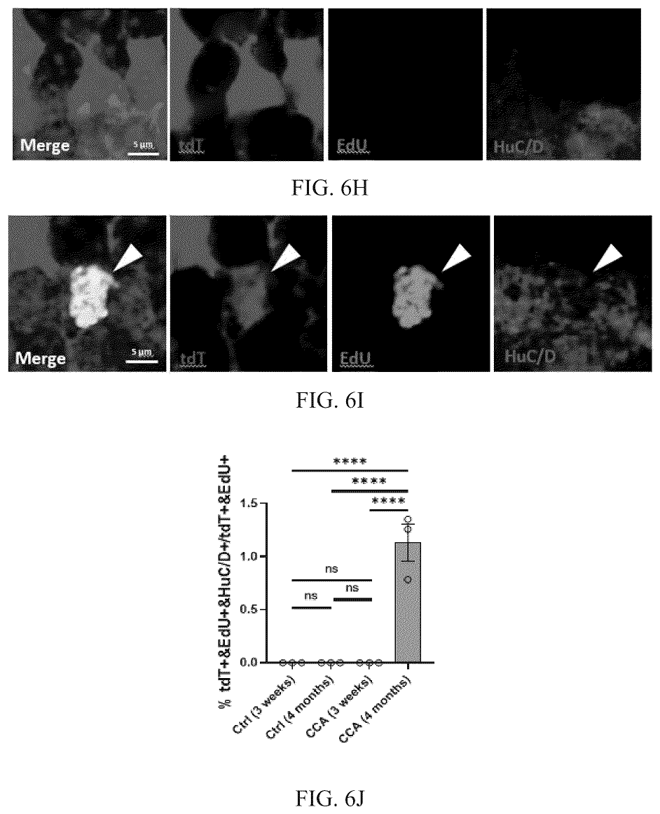

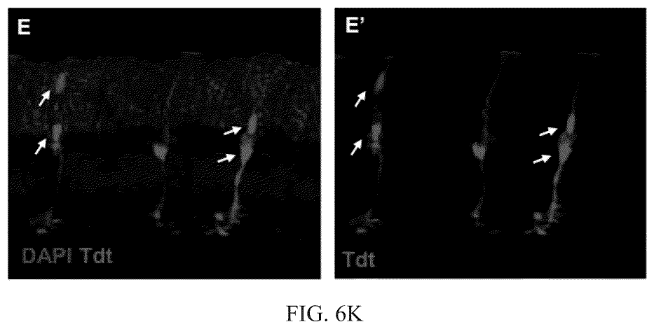

To determine whether ONL MG can differentiate into mature rods given an extended period, the morphology of EdU+MG in the ONL is examined. Despite extensive investigation, no EdU+Tdt+ cells in the ONL with rod outer segments (ROS), crucial for rod phototransduction, are identified (FIG. 6A and FIG. 6B). Additionally, the majority of ONL MG nuclei do not resemble rod nuclei. Furthermore, MG sparsely labeled with a low dose of Tamoxifen before CCA treatment shows that most ONL MG daughter cells retain apical processes (FIG. 6K). The mRNA expression analysis of rod and MG genes at 4 months after CCA treatment, including Rho, Gnat1, and Glul, does not show significant changes. Thus, it is concluded that ONL MG do not mature into rods despite ongoing rod gene expression, retaining their MG properties.

### Example 5. Few Bipolar and Amacrine-Like Cells are Regenerated from MG

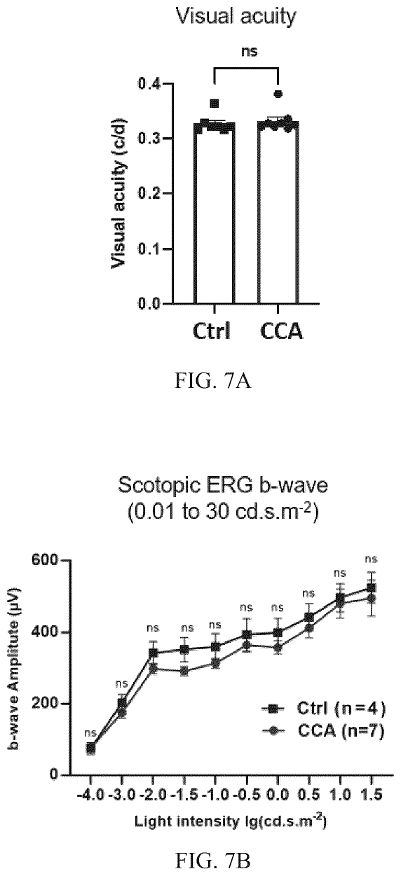

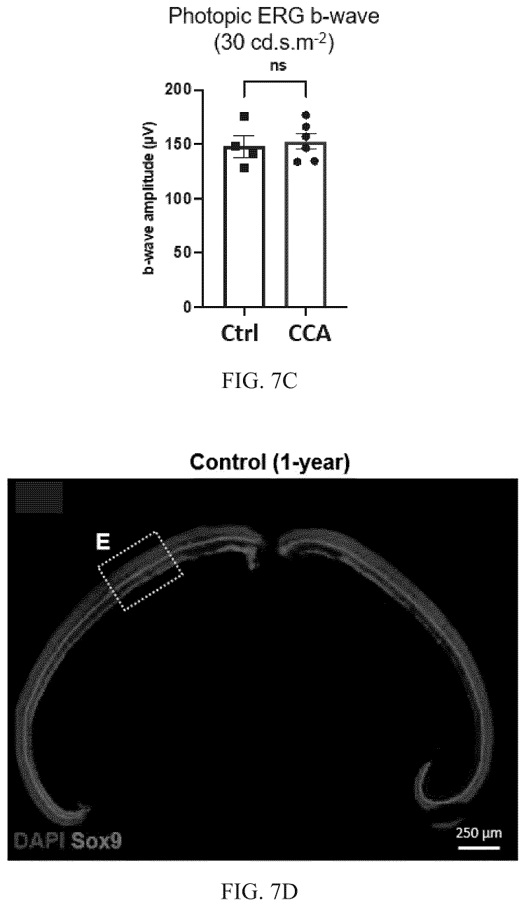

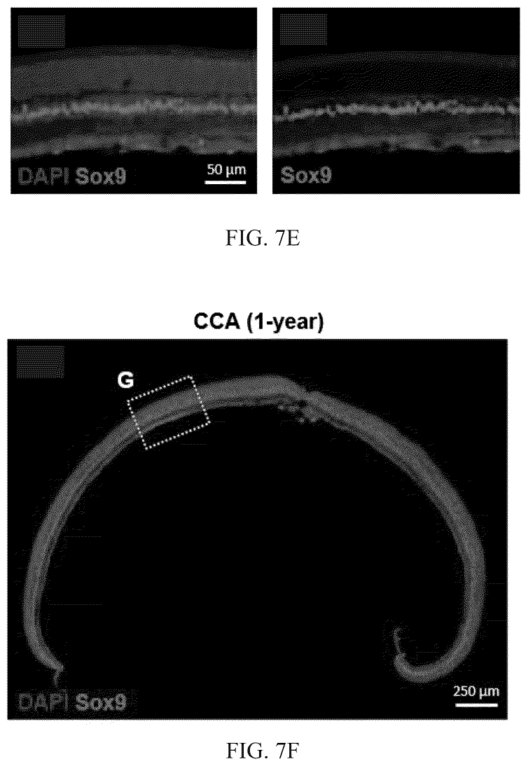

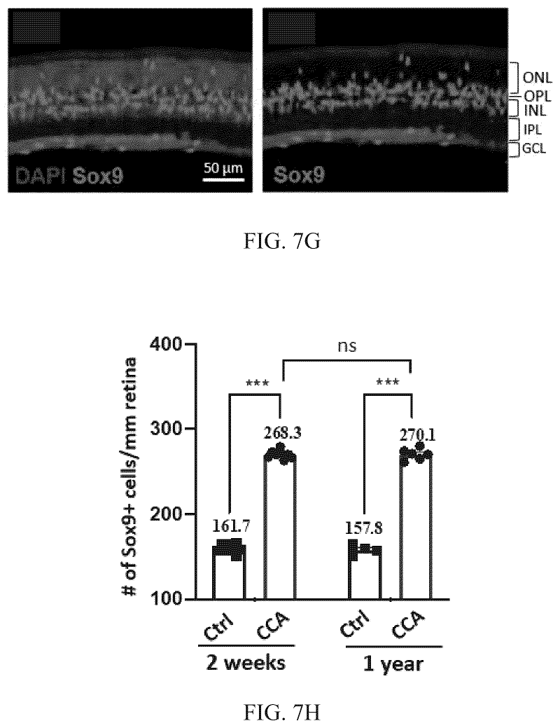

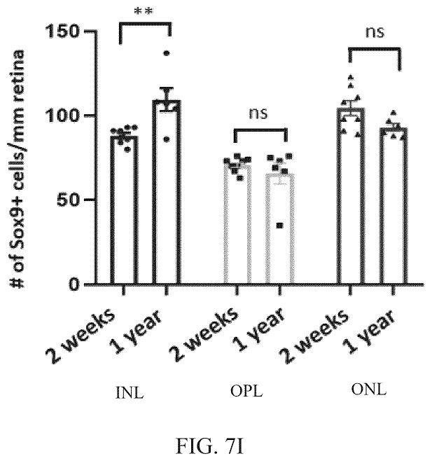

Bipolar (BP) and amacrine cells (AC) are two types of retinal neurons that have been demonstrated to regenerate from MG. Although scRNA-seq data does not reveal other retinal neuron-like clusters derived from MG after CCA treatment, the immunostaining is conducted for the bipolar marker Otx2 and the ganglion/amacrine marker HuC/D to explore whether rare MG-derived cells express these markers. To specifically identify de novo neurogenesis from MG and exclude any mislabeled neurons in the reporter mouse line, our analysis focused on EdU+Tdt+MG in Glast-Cre^{ERT2}; R26-tdTomato mice. At 4 months post-CCA treatment, it is observed that less than 1% of total EdU+Tdt+ cells express Otx2, whereas these cells are absent at 3 weeks post-CCA treatment (FIGS. 7A-7E). Rarely, HuC/D+EdU+Tdt+ cells are also found in the lower INL, where HuC/D+ amacrine cells are naturally located (FIGS. 7F-7J). Further investigation is required to determine whether these cells express other BP or AC markers and whether they function as interneurons connecting with the retinal circuitry. Nevertheless, these findings suggest that cell cycle re-activation alone enhances the plasticity of MG daughter cell fate, allowing some to differentiate into BP or AC-like neurons.

### Example 6. CCA does not Cause Retinal Dysplasia or Functional Deficit

To assess the long-term effects of CCA treatment, a cohort of C57BL/6 mice is monitored for one year following CCA injection (FIG. 7). Visual function assessments reveal no significant differences in visual acuity and electroretinography (ERG) between CCA-treated mice and WT mice one year after treatment (FIGS. 7A-7C). Upon examination of all harvested retinas from the CCA-treated group, no retinal tumors or dysplasia are observed. The retinal structure remains intact, showing no malignancies or disruptions in retinal layers or stratification (FIG. 7D and FIG. 7E). These results indicate that CCA treatment has no detrimental impact on retinal structure or function in mice, nor does it induce retinal tumors.

The MG marker Sox9 is utilized to assess the MG population in retinal sections harvested one-year post-CCA treatment. In control retinas, Sox9+MG cells are predominantly located in the INL (FIGS. 7A-7D). In contrast, CCA-treated retinas exhibit a significant expansion in the number of Sox9+MG cells, with approximately half of these cells residing in the ONL and OPL (FIGS. 7A-7D). Nevertheless, a substantial number of Sox9+MG cells remain in the INL, contributing to retinal homeostasis and explaining the observed preservation of retinal structure and function in CCA-treated eyes. Comparison of the numbers of Sox9+ cells after one year and two weeks of CCA treatment shows similar total counts (FIG. 7E), indicating no significant reduction in MG numbers over time. To investigate the possibility of MG cell death, particularly among those displaced to the ONL and OPL following CCA treatment, TUNEL assays are performed on CCA-treated retinas and no increase in TUNEL+ cells is found (FIG. 7I). While the possibility of undetected cell deaths among MG-derived cells cannot be excluded, the substantial presence of MG in the ONL and OPL at one year post-CCA treatment suggests that these MG-derived cells persist without undergoing significant cell death or losing their MG identity.

In summary, the present invention demonstrates that cyclin D1 overexpression alone can promote MG proliferation and that the combination of cyclin D1 overexpression and p27^{Kip1} KD is the most potent in driving MG proliferation in the mouse retina without an injury stimulus.

The strategy to stimulate the MG cell cycle for retinal regeneration must be carefully weighed against the potential risk of tumorigenesis. p27^{Kip1} and cyclin D1 are critical regulators of the cell cycle, and any abnormalities in their expression may impact cell division, potentially leading to tumor development. Previous studies indicate that mice lacking p27^{Kip1} are approximately 30% larger but generally do not exhibit malignancies, except for spontaneously developing pituitary tumors. Cyclin D1 is frequently overexpressed in various human cancers, such as breast and respiratory tract tumors. The concern regarding retinal tumor formation due to CCA treatment is mitigated by the use of AAV vectors for in vivo gene transfer. The AAV episomal genome becomes diluted after cell division, reducing the risk of continuous cyclin D1 overexpression and p27^{Kip1} KD leading to tumor formation. Consistently, the rate of MG proliferation decreases gradually over time, and most MG cells do not undergo multiple cell divisions. Additionally, the retinal structure remains normal without dysplasia. These results suggest that CCA is a potent and viable treatment to stimulate MG proliferation and may be a promising approach for use in combination with other neurogenic factors to promote MG-mediated retinal regeneration.

Furthermore, the upregulation of rod genes in MG residing in the ONL is an unexpected outcome of MG cell cycle activation by CCA. One hypothesis is that Cyclin D1 or p27Kip1 regulates rod gene expression in addition to their well-characterized functions in cell cycle regulation. Namely, besides cell cycle activation, Cyclin D1 overexpression and/or p27^{Kip1} KD may directly or indirectly promote rod gene expression, potentially explaining the rod-like MG population observed following CCA treatment.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of ±10%, ±5%, ±1%, or ±0.5% of the given value or range. The range may be indicated herein as from one endpoint to another endpoint or between two endpoints. Unless otherwise specified, all the ranges disclosed in the present disclosure include endpoints. When reference is made to “substantially” the same numerical value or characteristic, the term may refer to a value within ±10%, ±5%, ±1%, or ±0.5% of the average of the values.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A method of treating retinal degenerative diseases in a subject in need thereof, comprising:
administering an agonist selectively elevates the expression of cyclin D1 in Müller glia (MG) cells; and
administering an antagonist selectively knocks down the expression of p27^{kip1} in MG cells.

2. The method of claim 1, wherein the administration enables delivery to the subject's subretinal space or vitreous.

3. The method of claim 1, wherein the combination of cyclin D1 overexpression and p27^{kip1} knockdown drives MG proliferation and induces partial differentiation towards neuron-like MG cells.

4. The method of claim 1, wherein the retinal degenerative diseases comprise diabetic retinopathy, age-related macular degeneration (AMD), retinitis pigmentosa, Stargardt's disease, macular hole, and bright light-induced retina damage.

5. A composition for treating retinal degenerative diseases in a subject in need thereof, comprising an agonist selectively elevates the expression of cyclin D1 in MG cells, an antagonist selectively knocks down the expression of p27^{kip1} in MG cells and a pharmaceutically acceptable addition.

6. The composition of claim 5, wherein the agonist and the antagonist are integrated in a gene therapy technique, comprising a recombinant adeno-associated virus (rAAV), a recombinant adenovirus, a recombinant lentivirus, a recombinant retrovirus, a recombinant vaccinia virus, a recombinant herpes simplex virus, a naked plasmid, a lipid nanoparticle, a peptide-based vector and a polymer-based vector.

7. The composition of claim 5, wherein the agonist and the antagonist are integrated in a gene therapy technique, comprising a rAAV for delivering gene therapy to a retinal region.

8. The composition of claim 7, wherein the rAAV comprises:
a promoter DNA sequence obtained from a human glial fibrillary acidic protein (GFAP) gene;
a DNA sequence encoding cyclin D1; and
a short hairpin RNA sequence targeting p27^{kip1} gene, wherein the short hairpin RNA sequence is encoded in the 3′ untranslated region (UTR) of the cyclin D1 gene.

9. The composition of claim 8, wherein the promoter DNA sequence is at least 85% identical to the DNA sequence of SEQ ID NO: 01.

10. The composition of claim 8, wherein the DNA sequence encoding cyclin D1 is at least 85% identical to the DNA sequence of SEQ ID NO: 02 and encodes a functional cyclin D1.

11. The composition of claim 8, wherein the short hairpin RNA sequence is at least 85% identical to the DNA sequence of SEQ ID NO: 03.

12. The composition of claim 8, wherein the rAAV vector stimulates MG cells to overexpress cyclin D1 and knock down the expression of p27^{Kip1} in the MG cells.

13. The composition of claim 5, wherein the pharmaceutically acceptable addition comprises one or more of an excipient, a stability additive, a carrier, a diluent, or a solubilizer.

14. The composition of claim 5, wherein the composition is formulated to an administration form that enables delivery to the subject's subretinal space or vitreous.

15. The composition of claim 14, wherein the administration form is selected from an immediate-release form or a controlled-release form.

16. The composition of claim 14, wherein the composition is delivered through an approach selected from an intravitreal injection, or a subretinal injection.

17. The composition of claim 15, the administration form comprises an injection form, a hydrogel form, an ultrasonic ocular drug delivery form, a drug-eluting implant form, a nanoparticle-mediated delivery, and an intravitreal microneedle form.

18. The composition of claim 5, wherein the composition is delivered in combination with other neurogenic factors to enhance retinal regeneration.

19. Use of the composition of claim 5 for treating a retinal degenerative disease in a subject in need thereof, comprising:
administering an amount effective of the composition to the subject in need thereof.

20. The use of claim 19, wherein the retinal degenerative disease comprises diabetic retinopathy, AMD, retinitis pigmentosa, Stargardt's disease, macular hole, and bright light-induced retina damage.
